(19) United States
(12) Patent Application Publication
(10) Pub. No.: US 2017/0185149 A1
(43) Pub. Date: Jun. 29, 2017

(54) Single sensor brain wave monitor

(71) Applicant: Intel Corporation, Santa Clara, CA (US)

(72) Inventors: Olufemi B. Oluwafemi, Hillsboro, OR (US); Esther J. Kim, San Jose, CA (US); Marissa E. Powers, Philadelphia, PA (US); Leila Tavabi, Hillsboro, OR (US); Alejandro Ibarra Von Borstel, Zapopan (MX); Hector A. Cordourier Maruri, Guadalajara (MX)

(73) Assignee: Intel Corporation, Santa Clara, CA (US)

(21) Appl. No.: 14/998,297

(22) Filed: Dec. 26, 2015

(51) Int. Cl. G06F 3/01; A61B 5/04; A61B 5/0482; A61B 5/00; A61B 5/0476

(52) U.S. Cl. CPC G06F 3/015; A61B 5/6814; A61B 5/6803; A61B 5/0476; A61B 5/0482; A61B 5/04012; A61B 5/746

(57) ABSTRACT

In one example a data processing platform comprising circuitry to receive an input from a single electroencephalography (EEG) sensor and a single reference sensor, detect an increase in brain wave activity in a predetermined frequency range, and in response to the increase in brain wave activity in the predetermined frequency range, to generate an alert signal. Other examples may be described.

### BACKGROUND

The subject matter described herein relates generally to the field of electronic devices and more particularly to a single-sensor brain wave monitor.

In some circumstances systems and techniques to implement brain wave monitoring using a single sensor may find utility, e.g., in brain-computer interface (BCI) applications used with wearable and/or portable electronic devices.

### BRIEF DESCRIPTION OF THE DRAWINGS

The detailed description is described with reference to the accompanying figures.

FIG. 1 is an illustration of sensor placement in brain wave monitoring in accordance with some examples in accordance with some examples.

FIGS. 2A and 2B are schematic illustrations of eyewear apparatus which may be used in conjunction with sensors to brain wave monitoring in accordance with some examples.

FIG. 3A is a schematic illustration of an earpiece which may be used in conjunction with sensors to brain wave monitoring in accordance with some examples.

FIG. 3B is a schematic illustration of headwear which may be used in conjunction with sensors to brain wave monitoring in accordance with some examples.

FIG. 4 is a high-level schematic illustration of a processing platform which may be adapted to implement brain wave monitoring in accordance with some examples in accordance with some examples.

FIG. 5 is a flowchart illustrating operations in a method to implement brain wave monitoring in accordance with some examples in accordance with some examples.

FIGS. 6-10 are schematic illustrations of electronic devices which may be adapted to implement brain wave monitoring in accordance with some examples in accordance with some examples.

### DETAILED DESCRIPTION

Described herein are exemplary systems and methods to implement brain wave monitoring in accordance with some examples. In the following description, numerous specific details are set forth to provide a thorough understanding of various examples. However, it will be understood by those skilled in the art that the various examples may be practiced without the specific details. In other instances, well-known methods, procedures, components, and circuits have not been illustrated or described in detail so as not to obscure the particular examples.

By way of background, a significant number of accidents are caused by distracted, fatigued, and/or day dreaming drivers, heavy equipment operators, and motorcyclists. Such accidents tend to happen when people are alone, i.e., when there is no one supervising or otherwise interacting with them. One potential way to address this problem is to use Brain Computer Interface (BCI) devices to attempt to monitor, and measure a person's brain state and to predict and provide feedback when a user's brain state indicates that the person may be sleeping, eyes closed, distracted, fatigued, or otherwise inattentive.

Existing BCI devices are obtrusive due in part to the high number of sensors used to extract meaningful data representative of mental states. Many BCI headsets are cumbersome, uncomfortable and cannot be worn without attracting unwanted attention. The subject matter described herein addresses these and other issues by providing techniques to implement brain wave monitoring using a single electroencephalography (EEG) sensor and one reference sensor. This allows for a brain wave monitoring to be performed in a compact and convenient form factor that people are accustomed to wearing, e.g., on the arm of a pair of glasses, on an earpiece/headset/headphones, on a helmet or a hat.

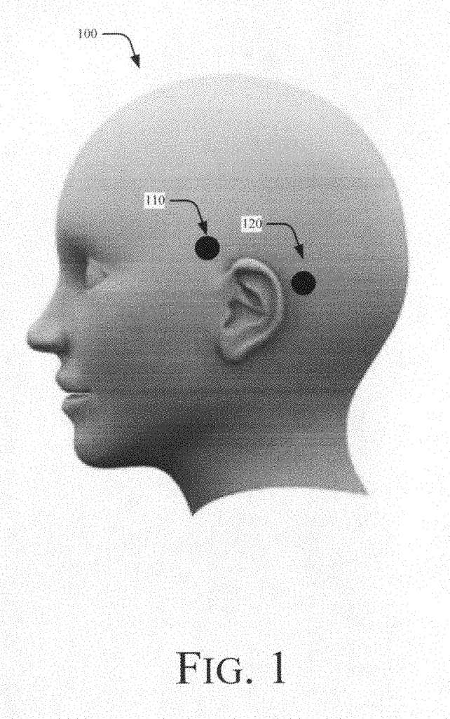

Further details will be described with reference to FIGS. 1-10.

FIG. 1 is an illustration of sensor placement in brain wave monitoring in accordance with some examples. Referring to FIG. 1, in accordance with principles described herein it has been determined that brain wave data capable of generating meaningful information about a person's brain state may be collected using a single EEG sensor 110 positioned proximate a temple region and forward of the ear of a user 100 and a single reference sensor 120 positioned behind an ear of the user 100. The EEG sensor 110 may be adapted to detect brain waves in a frequency range between 1 Hz and 20 Hz, and more particularly between a frequency range between 8 Hz and 12 Hz. The reference sensor 120 provides a ground reference for the EEG sensor 110. In various examples, the EEG sensor 110 and the reference sensor 120 may be positioned on only one side of the head of a user 100, or on both sides of the head of a user 100.

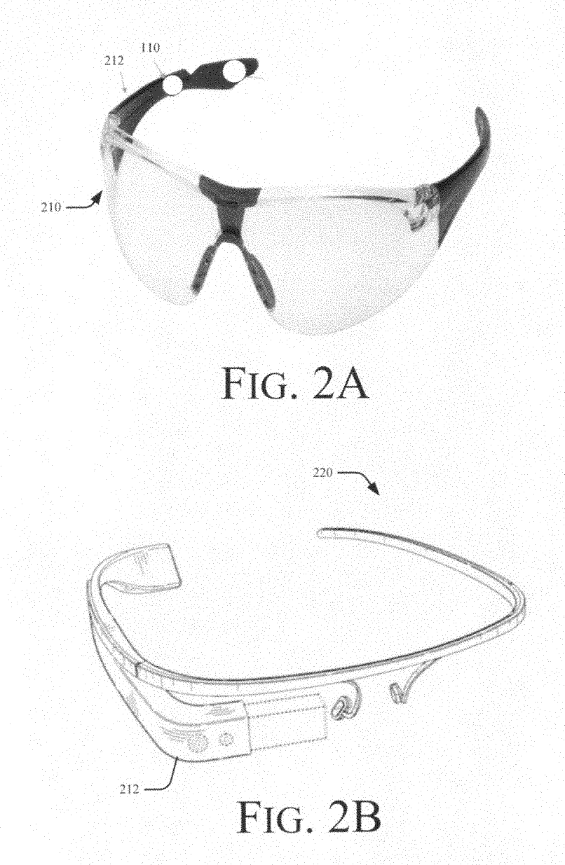

Referring to FIGS. 2A-2B, in some examples the single EEG sensor 110 and the single reference sensor 120 may be mounted on an arm 212 of a pair of glasses 210 and such that the EEG sensor 110 is positioned proximate a temple region and forward of the ear of a user 100 and a single reference sensor 120 positioned behind an ear of the user 100 when the user wears the glasses 210. In the example depicted in FIG. 2A the glasses 210 are safety glasses. In the example depicted in FIG. 2B the glasses 220 may include a wearable computing device 212 that includes a processing platform.

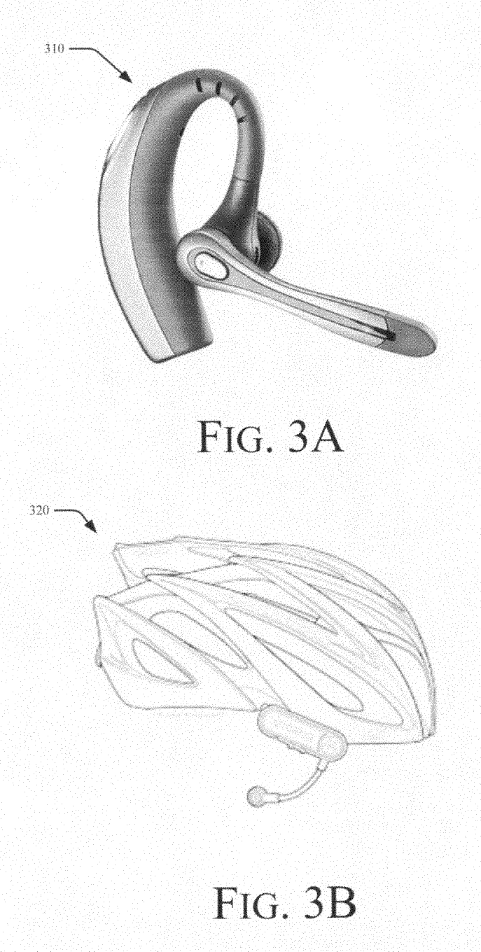

In other examples the EEG sensor 110 and reference sensor 120 may be mounted on other wearable device, e.g., an earpiece 310 as illustrated in FIG. 3A, or a helmet 320 as illustrated in FIG. 3B. In further examples the EEG sensor 110 and reference sensor 120 may be incorporated into other wearable devices such as a headband, a hat, or the like. In further examples the EEG sensor 110 and the reference sensor 120 may be implanted subcutaneously.

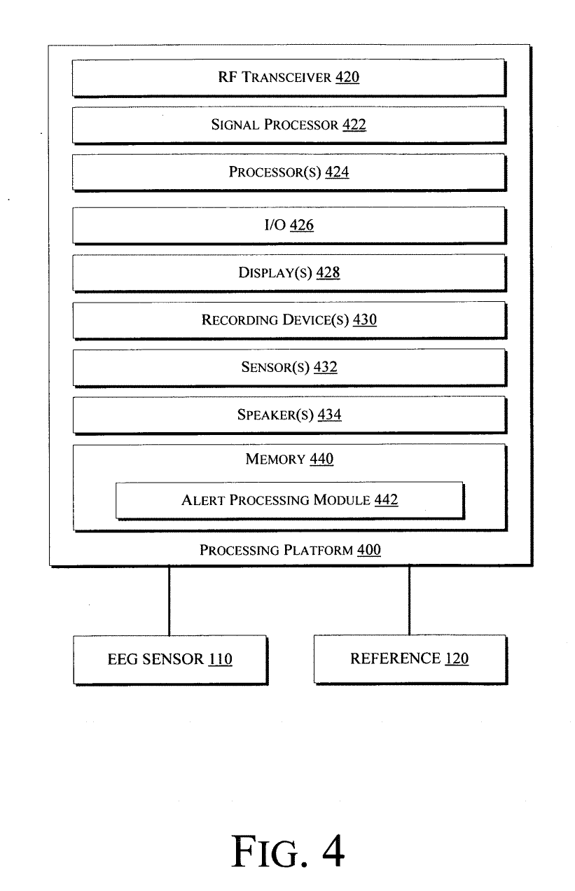

FIG. 4 is a schematic illustration of components of a processing platform 400 which may be adapted to implement brain wave monitoring in accordance with some examples. In some aspects processing platform 400 may be integrated into a wearable device such as a pair of glasses, an earpiece, a helmet, a headband, or the like. The specific implementation of the processing platform 200 is not critical. In one example the processing platform 400 may be implemented as an Intel® Curie™ platform.

In some examples processing platform 400 may include an RF transceiver 420 to transceive RF signals and a signal processing module 422 to process signals received by RF transceiver 420. RF transceiver 120 may implement a local wireless connection via a protocol such as, e.g., Bluetooth or 802.11X. IEEE 802.11a, b or g-compliant interface (see, e.g., IEEE Standard for IT-Telecommunications and information exchange between systems LAN/MAN—Part II: Wireless LAN Medium Access Control (MAC) and Physical Layer (PHY) specifications Amendment 4: Further Higher Data Rate Extension in the 2.4 GHz Band, 802.11G-2003). Another example of a wireless interface would be a general packet radio service (GPRS) interface (see, e.g., Guidelines on GPRS Handset Requirements, Global System for Mobile Communications/GSM Association, Ver. 3.0.1, December 2002).

Processing platform 400 may further include one or more processors 424 and memory 440. As used herein, the term “processor” means any type of computational element, such as but not limited to, a microprocessor, a microcontroller, a complex instruction set computing (CISC) microprocessor, a reduced instruction set (RISC) microprocessor, a very long instruction word (VLIW) microprocessor, or any other type of processor or processing circuit. In some examples, processor 424 may be one or more processors in the family of processors available from Intel® Corporation of Santa Clara, Calif. Alternatively, other processors may be used, such as Intel's Itanium®, XEON™, ATOM™, and Celeron® processors. Also, one or more processors from other manufactures may be utilized. Moreover, the processors may have a single or multi core design.

In some examples, memory 440 includes random access memory (RAM); however, memory module 440 may be implemented using other memory types such as dynamic RAM (DRAM), synchronous DRAM (SDRAM), and the like. Memory 440 may comprise one or more applications which execute on the processor(s) 424.

Processing platform 400 may further include one or more input/output devices 426 such as, e.g., a touchpad, microphone, or the like, and one or more displays 428, speakers 434, and one or more recording devices 430. By way of example, recording device(s) 430 may comprise one or more cameras and/or microphones

Processing platform may include one or more sensors 432 adapted to detect at least one of an acceleration, an orientation, or a position of the sensor, or combinations thereof. For example, sensors 432 may comprise one or more accelerometers, gyroscopes, magnetometers, piezoelectric sensors, or the like.

In some examples an alert processing module 442 may reside in memory 440 of processing platform 400. Alert processing module 442 may be embodied as logic instructions which, when executed on a processor, such as processor 424, configure the processing platform to perform operations for brain wave monitoring.

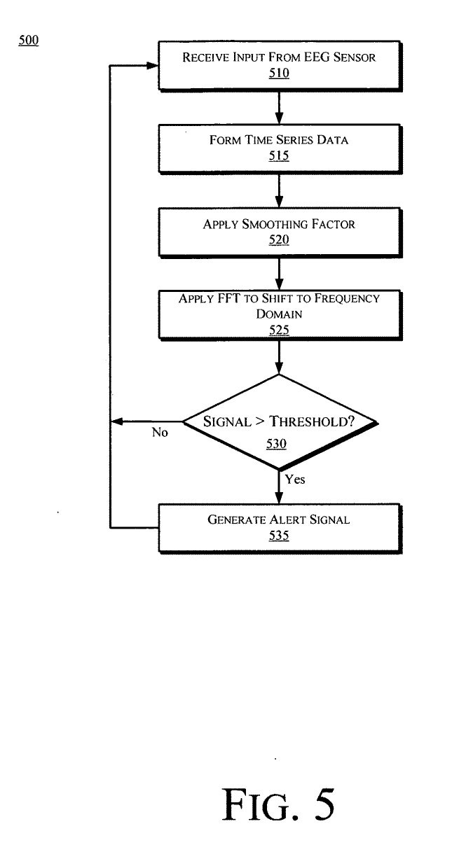

FIG. 5 is a flowchart illustrating operations in a method to implement brain wave monitoring in accordance with some examples in accordance with some examples. Referring to FIG. 5, at operation 510 input is received from EEG sensor 110. In some examples the input from the EEG sensor may be a voltage differential between the EEG sensor 110 and the reference sensor 120. At operation 515 the alert processing module 442 constructs a time series data set from the voltage differential detected by the EEG sensor 110.

Optionally, at operation 520 the alert processing module 442 may apply a smoothing factor to the time series data. By way of example, a smoothing factor may filter high voltage artifacts such as eye blinking.

At operation 525 the alert processing module 442 applies a time-frequency analysis (using a frequency range of interest) to the time series data set. This enables us to detect changes in the frequency band of interest as time progresses.

At operation 530 the alert processing module determines whether any portion of the received signal exceeds a threshold value and stays above the value for sometime (e.g., between one and two seconds). In some examples the threshold value may be a static threshold value determined by a manufacturer or distributor of the processing platform 400. In other examples the threshold value may be determined dynamically, e.g., using a machine/training process that is adapted for the particular user. By way of example, brain waves in the alpha band, which are between approximately 8 Hz and 12 Hz, tend to spike when a user enters a relaxed state in which the eyes are closed. Thus, the EEG sensor may determine whether a threshold value of brain waves in the alpha band exceed a threshold value.

If, at operation 530 the signal from the EEG does not exceed the threshold then control passes back to operation 410 and the alert processing module 442 continues to monitor input from the EEG sensor 110. By contrast, if at operation 530 the signal from the EEG does not exceed the threshold then control passes to operation 535 and the alert processing module 442 generates an alert signal. In some examples the alert signal may cause an audible alarm to be presented on the speaker(s) 434 and/or a visual alarm to be presented on a display 428 of the processing platform. In examples in which the processing platform 400 comprises (or is coupled to) a vibrator assembly the alert signal may activate the vibrator assembly.

Thus, the structures and operations described herein enable a single EEG sensor, in cooperation with a processing platform which may be incorporated into a wearable device, to implement brain wave monitoring operations. In some examples inputs from the single EEG sensor are used to determine whether a user is in a relaxed state which is, in turn, may trigger the processing platform to generate an alarm.

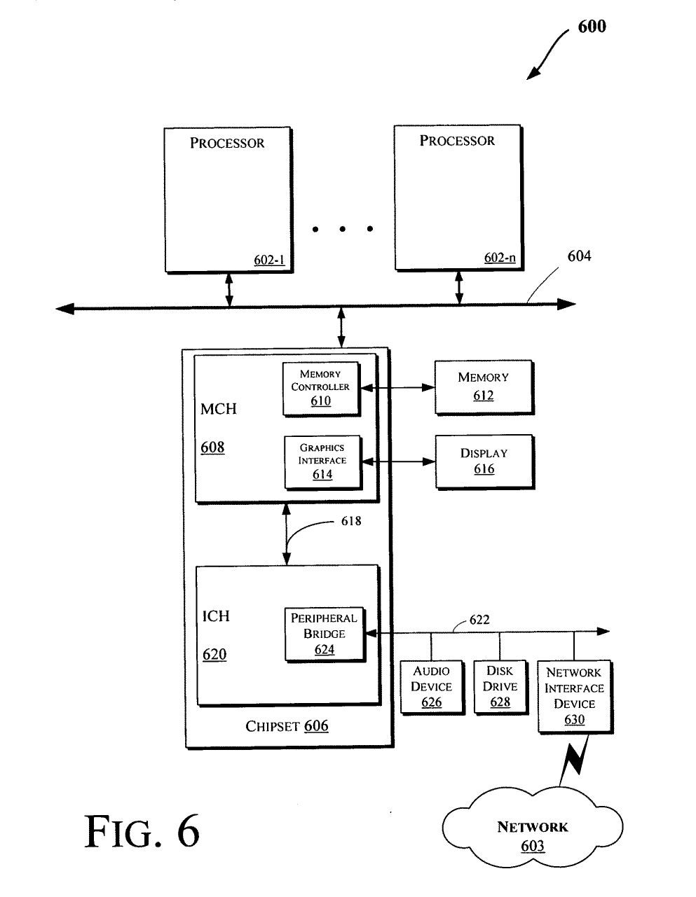

As described above, in some examples the electronic device may be embodied as a computer system. FIG. 6 illustrates a block diagram of a computing system 600 in accordance with an example. The computing system 600 may include one or more central processing unit(s) 602 or processors that communicate via an interconnection network (or bus) 604. The processors 602 may include a general purpose processor, a network processor (that processes data communicated over a computer network 603), or other types of a processor (including a reduced instruction set computer (RISC) processor or a complex instruction set computer (CISC)). Moreover, the processors 602 may have a single or multiple core design. The processors 602 with a multiple core design may integrate different types of processor cores on the same integrated circuit (IC) die. Also, the processors 602 with a multiple core design may be implemented as symmetrical or asymmetrical multiprocessors.

A chipset 606 may also communicate with the interconnection network 604. The chipset 606 may include a memory control hub (MCH) 608. The MCH 608 may include a memory controller 610 that communicates with a memory 612. The memory 612 may store data, including sequences of instructions, that may be executed by the processor 602, or any other device included in the computing system 600. In one example, the memory 612 may include one or more volatile storage (or memory) devices such as random access memory (RAM), dynamic RAM (DRAM), synchronous DRAM (SDRAM), static RAM (SRAM), or other types of storage devices. Nonvolatile memory may also be utilized such as a hard disk. Additional devices may communicate via the interconnection network 604, such as multiple processor(s) and/or multiple system memories.

The MCH 608 may also include a graphics interface 614 that communicates with a display device 616. In one example, the graphics interface 614 may communicate with the display device 616 via an accelerated graphics port (AGP). In an example, the display 616 (such as a flat panel display) may communicate with the graphics interface 614 through, for example, a signal converter that translates a digital representation of an image stored in a storage device such as video memory or system memory into display signals that are interpreted and displayed by the display 616. The display signals produced by the display device may pass through various control devices before being interpreted by and subsequently displayed on the display 616.

A hub interface 618 may allow the MCH 608 and an input/output control hub (ICH) 620 to communicate. The ICH 620 may provide an interface to I/O device(s) that communicate with the computing system 600. The ICH 620 may communicate with a bus 622 through a peripheral bridge (or controller) 624, such as a peripheral component interconnect (PCI) bridge, a universal serial bus (USB) controller, or other types of peripheral bridges or controllers. The bridge 624 may provide a data path between the processor 602 and peripheral devices. Other types of topologies may be utilized. Also, multiple buses may communicate with the ICH 620, e.g., through multiple bridges or controllers. Moreover, other peripherals in communication with the ICH 620 may include, in various examples, integrated drive electronics (IDE) or small computer system interface (SCSI) hard drive(s), USB port(s), a keyboard, a mouse, parallel port(s), serial port(s), floppy disk drive(s), digital output support (e.g., digital video interface (DVI)), or other devices.

The bus 622 may communicate with an audio device 626, one or more disk drive(s) 628, and a network interface device 630 (which is in communication with the computer network 603). Other devices may communicate via the bus 622. Also, various components (such as the network interface device 630) may communicate with the MCH 608 in some examples. In addition, the processor 602 and one or more other components discussed herein may be combined to form a single chip (e.g., to provide a System on Chip (SOC)). Furthermore, the graphics accelerator 616 may be included within the MCH 608 in other examples.

Furthermore, the computing system 600 may include volatile and/or nonvolatile memory (or storage). For example, nonvolatile memory may include one or more of the following: read-only memory (ROM), programmable ROM (PROM), erasable PROM (EPROM), electrically EPROM (EEPROM), a disk drive (e.g., 628), a floppy disk, a compact disk ROM (CD-ROM), a digital versatile disk (DVD), flash memory, a magneto-optical disk, or other types of nonvolatile machine-readable media that are capable of storing electronic data (e.g., including instructions).

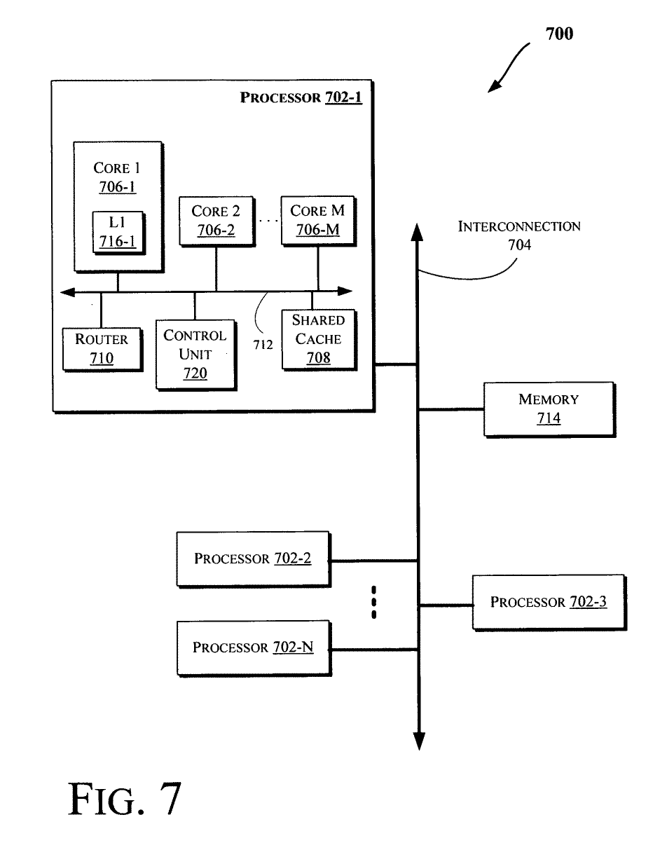

FIG. 7 illustrates a block diagram of a computing system 700, according to an example. The system 700 may include one or more processors 702-1 through 702-N (generally referred to herein as “processors 702” or “processor 702”). The processors 702 may communicate via an interconnection network or bus 704. Each processor may include various components some of which are only discussed with reference to processor 702-1 for clarity. Accordingly, each of the remaining processors 702-2 through 702-N may include the same or similar components discussed with reference to the processor 702-1.

In an example, the processor 702-1 may include one or more processor cores 706-1 through 706-M (referred to herein as “cores 706” or more generally as “core 706”), a shared cache 708, a router 710, and/or a processor control logic or unit 720. The processor cores 706 may be implemented on a single integrated circuit (IC) chip. Moreover, the chip may include one or more shared and/or private caches (such as cache 708), buses or interconnections (such as a bus or interconnection network 712), memory controllers, or other components.

In one example, the router 710 may be used to communicate between various components of the processor 702-1 and/or system 700. Moreover, the processor 702-1 may include more than one router 710. Furthermore, the multitude of routers 710 may be in communication to enable data routing between various components inside or outside of the processor 702-1.

The shared cache 708 may store data (e.g., including instructions) that are utilized by one or more components of the processor 702-1, such as the cores 706. For example, the shared cache 708 may locally cache data stored in a memory 714 for faster access by components of the processor 702. In an example, the cache 708 may include a mid-level cache (such as a level 2 (L2), a level 3 (L3), a level 4 (L4), or other levels of cache), a last level cache (LLC), and/or combinations thereof. Moreover, various components of the processor 702-1 may communicate with the shared cache 708 directly, through a bus (e.g., the bus 712), and/or a memory controller or hub. As shown in FIG. 7, in some examples, one or more of the cores 706 may include a level 1 (L1) cache 716-1 (generally referred to herein as “L1 cache 716”).

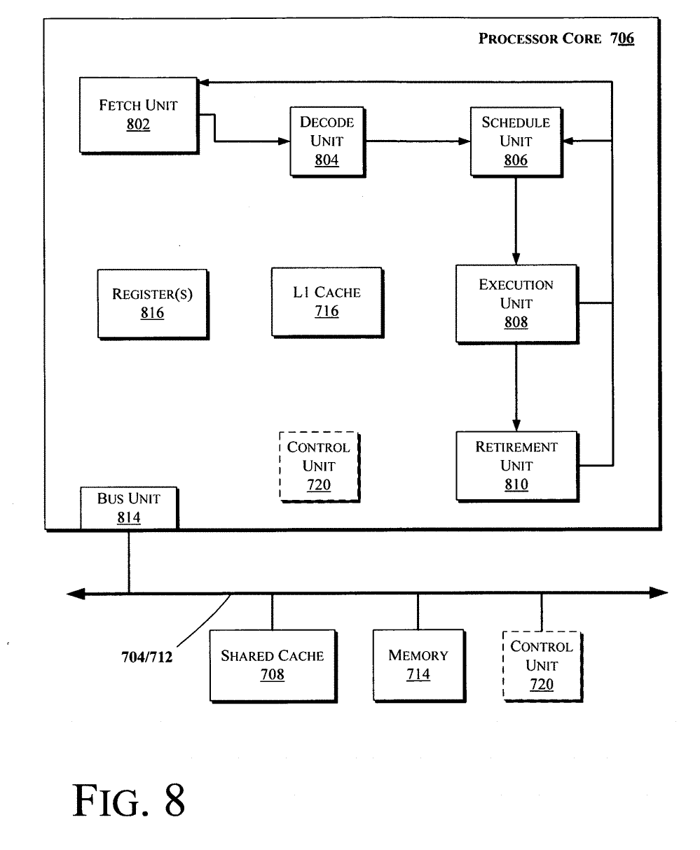

FIG. 8 illustrates a block diagram of portions of a processor core 706 and other components of a computing system, according to an example. In one example, the arrows shown in FIG. 8 illustrate the flow direction of instructions through the core 706. One or more processor cores (such as the processor core 706) may be implemented on a single integrated circuit chip (or die) such as discussed with reference to FIG. 7. Moreover, the chip may include one or more shared and/or private caches (e.g., cache 708 of FIG. 7), interconnections (e.g., interconnections 704 and/or 112 of FIG. 7), control units, memory controllers, or other components.

As illustrated in FIG. 8, the processor core 706 may include a fetch unit 802 to fetch instructions (including instructions with conditional branches) for execution by the core 706. The instructions may be fetched from any storage devices such as the memory 714. The core 706 may also include a decode unit 804 to decode the fetched instruction. For instance, the decode unit 804 may decode the fetched instruction into a plurality of uops (micro-operations).

Additionally, the core 706 may include a schedule unit 806. The schedule unit 806 may perform various operations associated with storing decoded instructions (e.g., received from the decode unit 804) until the instructions are ready for dispatch, e.g., until all source values of a decoded instruction become available. In one example, the schedule unit 806 may schedule and/or issue (or dispatch) decoded instructions to an execution unit 808 for execution. The execution unit 808 may execute the dispatched instructions after they are decoded (e.g., by the decode unit 804) and dispatched (e.g., by the schedule unit 806). In an example, the execution unit 808 may include more than one execution unit. The execution unit 808 may also perform various arithmetic operations such as addition, subtraction, multiplication, and/or division, and may include one or more an arithmetic logic units (ALUs). In an example, a co-processor (not shown) may perform various arithmetic operations in conjunction with the execution unit 808.

Further, the execution unit 808 may execute instructions out-of-order. Hence, the processor core 706 may be an out-of-order processor core in one example. The core 706 may also include a retirement unit 810. The retirement unit 810 may retire executed instructions after they are committed. In an example, retirement of the executed instructions may result in processor state being committed from the execution of the instructions, physical registers used by the instructions being de-allocated, etc.

The core 706 may also include a bus unit 714 to enable communication between components of the processor core 706 and other components (such as the components discussed with reference to FIG. 8) via one or more buses (e.g., buses 804 and/or 812). The core 706 may also include one or more registers 816 to store data accessed by various components of the core 706 (such as values related to power consumption state settings).

Furthermore, even though FIG. 7 illustrates the control unit 720 to be coupled to the core 706 via interconnect 812, in various examples the control unit 720 may be located elsewhere such as inside the core 706, coupled to the core via bus 704, etc.

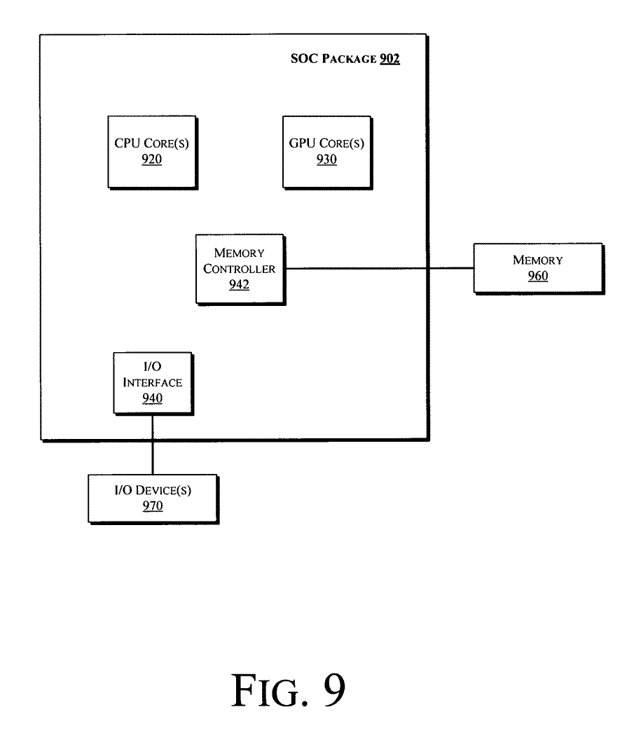

In some examples, one or more of the components discussed herein can be embodied as a System On Chip (SOC) device. FIG. 9 illustrates a block diagram of an SOC package in accordance with an example. As illustrated in FIG. 9, SOC 902 includes one or more processor cores 920, one or more graphics processor cores 930, an Input/Output (I/O) interface 940, and a memory controller 942. Various components of the SOC package 902 may be coupled to an interconnect or bus such as discussed herein with reference to the other figures. Also, the SOC package 902 may include more or less components, such as those discussed herein with reference to the other figures. Further, each component of the SOC package 902 may include one or more other components, e.g., as discussed with reference to the other figures herein. In one example, SOC package 902 (and its components) is provided on one or more Integrated Circuit (IC) die, e.g., which are packaged into a single semiconductor device.

As illustrated in FIG. 9, SOC package 902 is coupled to a memory 960 (which may be similar to or the same as memory discussed herein with reference to the other figures) via the memory controller 942. In an example, the memory 960 (or a portion of it) can be integrated on the SOC package 902.

The I/O interface 940 may be coupled to one or more I/O devices 970, e.g., via an interconnect and/or bus such as discussed herein with reference to other figures. I/O device(s) 970 may include one or more of a keyboard, a mouse, a touchpad, a display, an image/video capture device (such as a camera or camcorder/video recorder), a touch surface, a speaker, or the like.

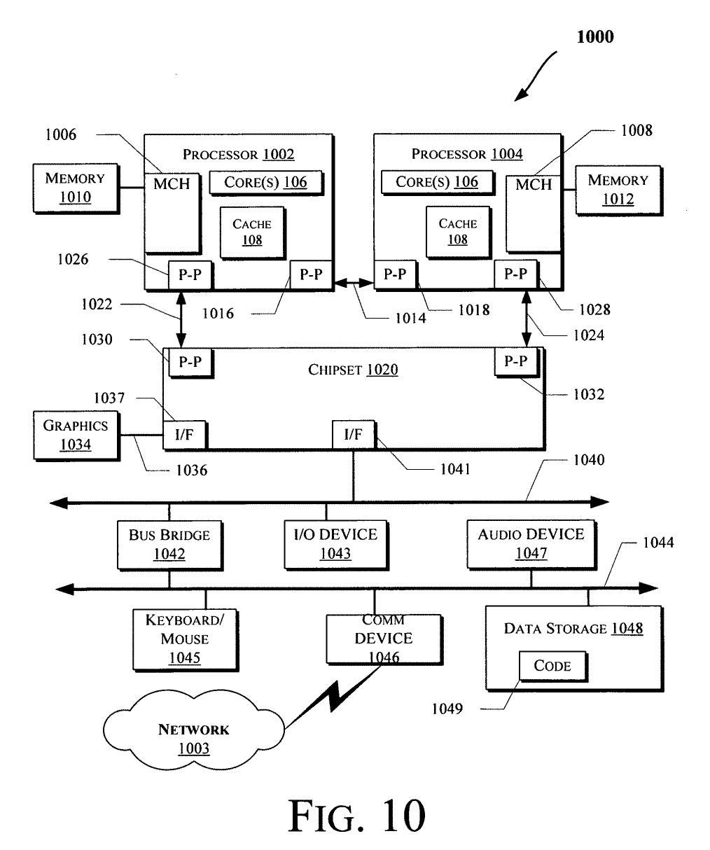

FIG. 10 illustrates a computing system 1000 that is arranged in a point-to-point (PtP) configuration, according to an example. In particular, FIG. 10 shows a system where processors, memory, and input/output devices are interconnected by a number of point-to-point interfaces. As illustrated in FIG. 10, the system 1000 may include several processors, of which only two, processors 1002 and 1004 are shown for clarity. The processors 1002 and 1004 may each include a local memory controller hub (MCH) 1006 and 1008 to enable communication with memories 1010 and 1012.

In an example, the processors 1002 and 1004 may be one of the processors 702 discussed with reference to FIG. 7. The processors 1002 and 1004 may exchange data via a point-to-point (PtP) interface 1014 using PtP interface circuits 1016 and 1018, respectively. Also, the processors 1002 and 1004 may each exchange data with a chipset 1020 via individual PtP interfaces 1022 and 1024 using point-to-point interface circuits 1026, 1028, 1030, and 1032. The chipset 1020 may further exchange data with a high-performance graphics circuit 1034 via a high-performance graphics interface 1036, e.g., using a PtP interface circuit 1037.

The chipset 1020 may communicate with a bus 1040 using a PtP interface circuit 1041. The bus 1040 may have one or more devices that communicate with it, such as a bus bridge 1042 and I/O devices 1043. Via a bus 1044, the bus bridge 1043 may communicate with other devices such as a keyboard/mouse 1045, communication devices 1046 (such as modems, network interface devices, or other communication devices that may communicate with the computer network 1003), audio I/O device, and/or a data storage device 1048. The data storage device 1048 (which may be a hard disk drive or a NAND flash based solid state drive) may store code 1049 that may be executed by the processors 1004.

The following examples pertain to further examples.

Example 1 is a controller comprising logic, at least partially including hardware logic, configured to

In Example 2, the subject matter of Example 1 can optionally include an arrangement in which the.

In Example 3, the subject matter of any one of Examples 1-2 can optionally include logic further configured to.

In Example 4, the subject matter of any one of Examples 1-3 can optionally include logic further configured to.

In Example 5, the subject matter of any one of Examples 1-4 can optionally include logic further configured to.

In Example 6, the subject matter of any one of Examples 1-5 can optionally include logic further configured to.

In Example 7, the subject matter of any one of Examples 1-6 can optionally include logic further configured to.

In Example 8, the subject matter of any one of Examples 1-7 can optionally include logic further configured to.

Example 9 is an electronic device, comprising an audio input device, a communication interface, and a controller comprising logic, at least partially including hardware logic, configured to

In Example 10, the subject matter of Example 9 can optionally include an arrangement in which the logic includes.

In Example 11, the subject matter of any one of Examples 9-10 can optionally include logic further configured to.

In Example 12, the subject matter of any one of Examples 9-11 can optionally include logic further configured to.

In Example 13, the subject matter of any one of Examples 9-12 can optionally include logic further configured to.

In Example 14, the subject matter of any one of Examples 9-13 can optionally include logic further configured to.

In Example 15, the subject matter of any one of Examples 9-14 can optionally include logic further configured to.

In Example 16, the subject matter of any one of Examples 9-15 can optionally include logic further configured to.

Example 17 is an electronic device, comprising.

In Example 18 the subject matter of Example 17 can optionally include an arrangement in which the.

In Example 19 the subject matter of any one of Examples 17-18 can optionally include logic further configured to.

In Example 20 the subject matter of any one of Examples 17-19 can optionally include logic further configured to.

The terms “logic instructions” as referred to herein relates to expressions which may be understood by one or more machines for performing one or more logical operations. For example, logic instructions may comprise instructions which are interpretable by a processor compiler for executing one or more operations on one or more data objects. However, this is merely an example of machine-readable instructions and examples are not limited in this respect.

The terms “computer readable medium” as referred to herein relates to media capable of maintaining expressions which are perceivable by one or more machines. For example, a computer readable medium may comprise one or more storage devices for storing computer readable instructions or data. Such storage devices may comprise storage media such as, for example, optical, magnetic or semiconductor storage media. However, this is merely an example of a computer readable medium and examples are not limited in this respect.

The term “logic” as referred to herein relates to structure for performing one or more logical operations. For example, logic may comprise circuitry which provides one or more output signals based upon one or more input signals. Such circuitry may comprise a finite state machine which receives a digital input and provides a digital output, or circuitry which provides one or more analog output signals in response to one or more analog input signals. Such circuitry may be provided in an application specific integrated circuit (ASIC) or field programmable gate array (FPGA). Also, logic may comprise machine-readable instructions stored in a memory in combination with processing circuitry to execute such machine-readable instructions. However, these are merely examples of structures which may provide logic and examples are not limited in this respect.

Some of the methods described herein may be embodied as logic instructions on a computer-readable medium. When executed on a processor, the logic instructions cause a processor to be programmed as a special-purpose machine that implements the described methods. The processor, when configured by the logic instructions to execute the methods described herein, constitutes structure for performing the described methods. Alternatively, the methods described herein may be reduced to logic on, e.g., a field programmable gate array (FPGA), an application specific integrated circuit (ASIC) or the like.

In the description and claims, the terms coupled and connected, along with their derivatives, may be used. In particular examples, connected may be used to indicate that two or more elements are in direct physical or electrical contact with each other. Coupled may mean that two or more elements are in direct physical or electrical contact. However, coupled may also mean that two or more elements may not be in direct contact with each other, but yet may still cooperate or interact with each other.

Reference in the specification to “one example” or “some examples” means that a particular feature, structure, or characteristic described in connection with the example is included in at least an implementation. The appearances of the phrase “in one example” in various places in the specification may or may not be all referring to the same example.

Although examples have been described in language specific to structural features and/or methodological acts, it is to be understood that claimed subject matter may not be limited to the specific features or acts described. Rather, the specific features and acts are disclosed as sample forms of implementing the claimed subject matter.

What is claimed is:

1. A data processing platform comprising circuitry to:
receive an input from a single electroencephalography (EEG) sensor and a single reference sensor;
detect, based on the input from the single EEG sensor, an increase in brain wave activity in a predetermined frequency range; and
in response to the increase in brain wave activity in the predetermined frequency range, to generate an alert signal.

2. The data processing platform of claim 1, wherein the single EEG sensor is positioned proximate a temple region and forward of the ear of a user of the data processing platform and the single reference sensor is positioned behind an ear of the user.

3. The data processing platform of claim 2, wherein the single EEG sensor and the single reference sensor are mounted on at least one of:
an arm of a pair of glasses;
an earpiece;
a helmet;
a headband; or
a hat.

4. The data processing platform of claim 2, wherein the single EEG sensor and the single reference sensor are implanted subcutaneously.

5. The data processing platform of claim 4, wherein the predetermined frequency range is between 8 Hz and 12 Hz.

6. The data processing platform of claim 5, further comprising circuitry to:
form a time series data of brain activity data collected by the single EEG sensor.

7. The data processing platform of claim 6, further comprising circuitry to:
perform a fast Fourier transform (FFT) on the time series data.

8. The data processing platform of claim 7, further comprising circuitry to:
compare an amplitude of an EEG signal in the predetermined frequency range to a threshold amplitude.

9. The data processing platform of claim 8, wherein the threshold amplitude is adjusted dynamically over time for a specific user.

10. The data processing platform of claim 1 wherein, in response to the alert signal, the data processing platform activates at least one of:
an audio alarm;
a visual alarm; or
a vibrating alarm.

11. A method to monitor brain wave states, comprising:
receiving, in a processing platform, an input from a single electroencephalography (EEG) sensor and a single reference sensor;
detecting, in the processing platform, an increase in brain wave activity in a predetermined frequency range based on the input from the single EEG sensor; and
in response to the increase in brain wave activity in the predetermined frequency range, generating, in the processing platform, an alert signal.

12. The method of claim 11, wherein the single EEG sensor is positioned proximate a temple region and forward of the ear of a user of the data processing platform and the single reference sensor is positioned behind an ear of the user.

13. The method of claim 12, wherein the single EEG sensor and the single reference sensor are mounted on at least one of:
an arm of a pair of glasses;
an earpiece;
a helmet;
a headband; or
a hat.

14. The method of claim 12, wherein the single EEG sensor and the single reference sensor are implanted subcutaneously.

15. The method of claim 14, wherein the predetermined frequency range is between 8 Hz and 12 Hz.

16. The method of claim 15, further comprising:
forming a time series data of brain activity data collected by the single EEG sensor.

17. The method of claim 16, further comprising:
performing a fast Fourier transform (FFT) on the time series data.

18. The method of claim 17, further comprising:
comparing an amplitude of an EEG signal in the predetermined frequency range to a threshold amplitude.

19. The method of claim 18, wherein the threshold amplitude is adjusted dynamically over time for a specific user.

20. The method of claim 11 further comprising, in response to the alert signal, activating at least one of:
an audio alarm;
a visual alarm; or
a vibrating alarm.
